(19) United States
(12) Patent Application Publication
(10) Pub. No.: US 2011/0086086 A1
(43) Pub. Date: Apr. 14, 2011

(54) TRANSDERMAL SYSTEM FOR VARENICLINE

(75) Inventors: Barbara Alice Johnson, Niantic, CT (US); Carl Bernard Ziegler, East Lyme, CT (US)

(73) Assignee: Pfizer Inc

(21) Appl. No.: 11/574,045

(22) PCT Filed: Jun. 13, 2006
(86) PCT No.: PCT/IB06/02081

§ 371 Date: Feb. 21, 2007

Related U.S. Application Data
(60) Provisional application No. 60/702,961, filed on Jul. 26, 2005.

(51) Int. Cl. A61K 9/70; C07D 471/08; A61K 31/4985; A61P 25/34

(52) U.S. Cl. 424/449; 544/343; 514/250

(57) ABSTRACT

The invention provides transdermal compositions comprising varenicline or its pharmaceutically acceptable salt or prodrug form.

### FIELD OF THE INVENTION

The present invention relates to pharmaceutical compositions for medicinal uses thereof.

### BACKGROUND ART

Varenicline has the structure:

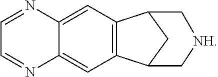

Varenicline is also known as 5,8,14-triazatetracyclo[10.3.1.0^{2,11}.0^{4,9}]-hexadeca-2(11),3,5,7,9-pentaene or 7,8,9,10-tetrahydro-6,10-methano-6H-pyrazino[2,3-h][3]-benzazepine. Varenicline and pharmaceutically acceptable acid addition salts thereof are referred to in International Patent Publication WO 99/35131, published Jul. 15, 1999, the contents of which are incorporated herein by reference.

Varenicline binds to neuronal nicotinic acetylcholine specific receptor sites and is useful in modulating cholinergic function. Accordingly, this compound is useful in the treatment of various conditions or diseases including, but not limited to, inflammatory bowel disease (including, but not limited to, ulcerative colitis, pyoderma gangrenosum and Crohn's disease), irritable bowel syndrome, spastic dystonia, chronic pain, acute pain, celiac sprue, pouchitis, vasoconstriction, anxiety, panic disorder, depression, bipolar disorder, autism, sleep disorders, jet lag, amyotrophic lateral sclerosis (ALS), cognitive dysfunction, hypertension, bulimia, anorexia, obesity, cardiac arrhythmias, gastric acid hypersecretion, ulcers, pheochromocytoma, progressive supranuclear palsy, chemical dependencies and addictions (e.g., dependencies on, or addictions to nicotine (and/or tobacco products), alcohol, benzodiazepines, barbiturates, opioids or cocaine), headache, migraine, stroke, traumatic brain injury (TBI), obsessive-compulsive disorder (OCD), psychosis, Huntington's chorea, tardive dyskinesia, hyperkinesia, dyslexia, schizophrenia, multi-infarct dementia, age-related cognitive decline, epilepsy, including petit mal absence epilepsy, senile dementia of the Alzheimer's type (AD), Parkinson's disease (PD), attention deficit hyperactivity disorder (ADHD) and Tourette's Syndrome.

Varenicline is a highly potent compound such that dosage forms are necessarily highly diluted with excipients. The excipients provide dosage forms with adequate stability, while also providing for such desirable features as controlling the drug dissolution (e.g., either fast dissolving or slow dissolving in a controlled-release system as described in co-pending applications U.S. Patent Publication No. 2003-0180360 A1, published Sep. 25, 2003, and Ser. No. 10/848,464, filed May 18, 2004, the contents of which are hereby incorporated by reference in their entirety), masking bad taste, and providing appropriate properties for preparation of the dosage form (i.e., compression properties for tablets). Finally, because of the high dilution with excipients, reactivity of varenicline with the excipients themselves or with trace impurities (i.e., degradants) of the excipients can be especially problematic.

There are advantages of delivering varenicline in the form of a transdermal composition. For example, relative to an oral dosage form such as a tablet or capsule delivery of varenicline via a transdermal composition would be a preferred choice by patients who have difficulty in swallowing tablets, capsules or other solids.

The tablet dosage form of varenicline has shown, in some instances, a certain level of nausea in patients. There is a need to reduce these side effects. A gradual release of the varenicline dosage form such as would be the case from a transdermal composition might prove to be useful towards reducing the incidence of nausea and enhance the desirability of the drug to a larger patient population requiring its use. Finally, it is likely to assume there would be a higher compliance rate if a patient could apply a transdermal patch that delivers therapeutically useful levels of active ingredient over the course of a day or a longer period of time versus taking a once or twice daily tablet or capsule with water. Accordingly, there is a need for providing transdermal dosage forms of varenicline.

### SUMMARY OF THE INVENTION

The present invention relates to the transdermal application of varenicline and its pharmaceutically acceptable salt forms, as well as any prodrugs thereof herein referred to as the active ingredient and methods for delivering it to an individual, in particular to such compositions in adhesive coated sheet form that are suitable for administration to skin to provide a local or systemic therapeutic effect. The composition comprises a backing sheet that is flexibly conformable to the area of skin, the backing sheet having opposing surfaces that are respectively distal and proximal to the skin when applied; and a coating on the proximal surface of the backing sheet. The coating comprises (a) an adhesive and (b) a water-soluble active agent comprising varenicline or salt thereof, the active agent being in a therapeutically effective total amount and dispersed in a matrix that comprises zero to less than an active agent solubilizing effective amount in total of one or more solvents other than the adhesive.

When one or more solvents other than the adhesive, for example water or a polyhydric alcohol such as polyethylene glycol or propylene glycol, are defined herein as present in the matrix at “less than an active agent solubilizing effective amount in total”, it will be understood that the amount of such solvents is insufficient to dissolve all of the active agent present in the coating. The active agent can be dispersed in solid particulate form in the matrix. Alternatively, where the matrix is formed predominantly by the adhesive, the active agent can optionally be wholly or partly molecularly dispersed, i.e., in solid solution, in such a matrix.

The invention also relates to processes for preparing such compositions and to methods of treatment comprising administration of such compositions to skin of a subject in need thereof.

The present compositions are not limited by any process used to prepare them. In an illustrative process, the active agent is dissolved in a solvent or mixture of solvents, for example ethanol and water, prior to mixing with the adhesive. However, typically the solvent or mixture of solvents is later removed by heating, thus the final composition, in accordance with the present invention, contains no such solvents or has less than an active agent solubilizing effective amount in total of such solvents.

In a preferred embodiment the coating comprises a layer having the active agent dispersed in a matrix that comprises the adhesive. Alternatively, the coating can comprise two layers: a reservoir layer that comprises the active agent adjacent to the backing sheet, and an adhesive layer that is proximal to the skin when applied. Optionally in such a coating, a membrane that permits passage of the active agent is present between the reservoir layer and the adhesive layer.

In preferred compositions, the coating further comprises one or more skin permeation enhancers. Preferably a peelable release liner is also provided. This liner, prior to use, is adjacent to the layer that contains the adhesive, and is removed prior to application of the composition to the skin.

Another embodiment is a method of making a pressure sensitive adhesive matrix patch for transdermal delivery using the active ingredient. In a specific embodiment for transdermal administration, the active ingredient can be contained in a matrix from which it is released in the desired gradual, constant and controlled manner. The permeability of the matrix during the release of the active ingredient is based on diffusion. There is further provided a method of systemic treatment of a subject, the method comprising applying a pharmaceutical composition as provided herein to a skin surface of the subject, and leaving the composition in place for a time period effective to permit transdermal delivery of a therapeutic amount of the active agent.

### DETAILED DESCRIPTION OF THE INVENTION

The present invention relates to the transdermal application of varenicline and its pharmaceutically acceptable salt forms as well as any prodrugs thereof, herein referred to as the active ingredient and methods for delivering it to an individual, in particular to such compositions in adhesive coated sheet form that are suitable for administration to skin to provide a local or systemic therapeutic effect. The present invention utilizes varenicline or its pharmaceutically acceptable salt as the active ingredient. Varenicline can be used per se or in the form of its pharmaceutically acceptable salt, solvate and/or hydrate. Although any pharmaceutically acceptable form of varenicline can be used in connection with the present invention, it is preferable to use a salt form of the drug. A particularly preferred salt form of the drug is the L-tartrate salt.

In particular, the present invention provides a method for reducing nicotine addiction or aiding in the cessation or lessening of tobacco use in a subject. The method includes steps of administering to a subject an amount of the varenicline that is effective in reducing nicotine addiction or aiding in the cessation or lessening of tobacco use via administration of a transdermal dosage form of the drug.

The present invention can be used to treat disorders or conditions including, but not limited to, inflammatory bowel disease, ulcerative colitis, pyoderma gangrenosum, Crohn's disease, irritable bowel syndrome, spastic dystonia, chronic pain, acute pain, celiac sprue, pouchitis, vasoconstriction, anxiety, panic disorder, depression, bipolar disorder, autism, sleep disorders, jet lag, amyotrophic lateral sclerosis (ALS), cognitive dysfunction, hypertension, bulimia, anorexia, obesity, cardiac arrhythmias, gastric acid hypersecretion, ulcers, pheochromocytoma, progressive supranuclear palsy, chemical dependencies and addictions; dependencies on, or addictions to, nicotine, tobacco products, alcohol, benzodiazepines, barbiturates, opioids or cocaine; headache, stroke, traumatic brain injury (TBI), obsessive-compulsive disorder (OCD), psychosis, Huntington's Chorea, tardive dyskinesia, hyperkinesia, dyslexia, schizophrenia, multi-infarct dementia, age related cognitive decline, epilepsy, petit mal absence epilepsy, senile dementia of the Alzheimer's type (AD), Parkinson's disease (PD), attention deficit hyperactivity disorder (ADHD), Tourette's Syndrome, and any other similar disorder or condition known to those of skill in the art.

The term “varenicline,” as used herein means the drug that binds to neuronal nicotinic acetylcholine specific receptor sites, and is useful in modulating cholinergic function. Varenicline has the general formula of:

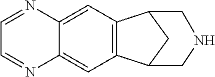

Varenicline includes the parent drug and all pharmaceutically acceptable salts and prodrugs thereof. The parent drug of varenicline is described in International Patent Publication WO 99/35131, published Jul. 15, 1999, the contents of which are incorporated herein by reference in their entirety. In any of the embodiments, varenicline or any of its pharmaceutically acceptable salts, solvates and/or hydrates can be used. Procedures for making varenicline are described in U.S. Pat. No. 6,410,550, the contents of which are incorporated herein by reference in their entirety. The resolution of racemic mixtures of varenicline is described in WO01/62736, which is also incorporated herein by reference in its entirety.

The term “mgA” refers to the number of milligrams of active drug based on the free base form of the drug.

The term “pharmaceutically acceptable” means the substance or composition must be compatible chemically, physically, and/or toxicologically, with the other components comprising a formulation, and/or the mammal being treated therewith.

The term “pharmaceutically acceptable salt” means non-toxic acid addition salts derived from inorganic and organic acids. Suitable salt derivatives include, but are not limited to, halides, thiocyanates, sulfates, bisulfates, sulfites, bisulfites, arylsulfonates, alkylsulfates, phosphonates, monohydrogen-phosphates, dihydrogenphosphates, metaphosphates, pyrophosphonates, alkanoates, cycloalkylalkanoates, arylalkonates, adipates, alginates, aspartates, benzoates, fumarates, glucoheptanoates, glycerophosphates, lactates, maleates, nicotinates, oxalates, palmitates, pectinates, picrates, pivalates, succinates, tartarates, citrates, camphorates, camphorsulfonates, digluconates, trifluoroacetates, and the like.

The term “active ingredient” means the therapeutically active compound, varenicline, as well as any prodrugs thereof and pharmaceutically acceptable salts, hydrates, and solvates of the compound and the prodrugs.

The term “ingredients” means any excipients, diluents, solvents, permeation enhancer, preservatives, buffers, gel forming agents, lubricants, carriers, stabilizers, gels, dyes, pigments, surfactants, inert fillers, tackifies, texturizers, softeners, emulsifiers, and mixtures thereof that are formulated with varenicline or any pharmaceutically acceptable salts, hydrates, and solvates of this drug.

The term “appropriate period of time” or “suitable period of time” means the period of time necessary to achieve a desired effect or result. For example, a mixture can be blended until a potency distribution is reached that is within an acceptable range for a given application or use of the blended mixture.

The term “unit dose,” “unit dosage,” or “unit dosage form” means a physically discrete unit that contains a predetermined quantity of active ingredient calculated to produce a desired therapeutic effect.

The term “effective amount,” as used herein means the amount determined by such considerations as are known in the art of reducing nicotine addiction or aiding in the cessation or lessening of tobacco use in an individual, wherein it must be effective to provide measurable relief in treated individuals such as exhibiting improvements including, but not limited to, more rapid recovery, improvement or elimination of symptoms or reduction of complications, lack of dependency upon nicotine-containing compounds, lack of desire towards nicotine-containing compounds, or other measurements as appropriate and known to those skilled in the medical arts.

The present invention has numerous embodiments. In any of the embodiments, pharmaceutical compositions of varenicline can be desirably administered in doses ranging from about 0.1 mgA up to about 6 mgA per day (where mgA refers to mg of active drug based on the free base form of the drug), more preferably from about 0.5 to 4 mgA/day, and most preferably from about 1 to 4 mgA per day in single or divided doses. Variations in such dosages, however, necessarily occur depending upon the weight and condition of the subject being treated. Depending on individual responses, dosage levels below the lower limit of the aforesaid range can be more than adequate, while in other cases still larger doses can be employed without causing any harmful side effects. The final pharmaceutical composition is processed into a unit dosage form and then packaged for distribution. The processing step varies depending upon the particular unit transdermal dosage form. Those of skill in the art are well aware of the procedures used for manufacturing the various unit dosage forms.

The present invention provides transdermal compositions containing the active ingredient and methods for delivering it to an individual. The active ingredient in the transdermal composition is gradually released into the systemic circulation through the skin. Moreover, it is reasonable to assume that the active ingredient within transdermal composition, is released through the skin over a suitable period of time. It is believed that the controlled release of the active ingredient from the transdermal composition prevents excessive concentrations of the drug in the body which in turn reduces or prevents the nausea side effect.

A pharmaceutical composition of the invention is described herein as an “adhesive coated sheet”, a generic term which will be understood to embrace patches, tapes, poultices, pads, plasters, cataplasms and dressings that are adhesive to skin. The components of the adhesive coated sheet are described herein with reference to a skin surface to which the composition is to be applied. As applied to a layer or surface herein, the term “proximal” means toward the skin surface and the term “distal” means away from the skin surface, when the composition is correctly applied.

The most distal layer of the composition is a backing sheet that is flexibly conformable to the skin surface. Any suitable material can be used for the backing sheet, but typically a polymer film, e.g., one comprising one or more of polyethylene, polyvinyl chloride, ethyl vinyl acetate, polyurethane and polyester, or a woven or nonwoven fabric, optionally having a polymer film laminated thereon, is used. The backing sheet can be airtight and/or waterproof, providing a substantially occlusive dressing. Alternatively, a backing sheet can be used having pores or other means for circulation of air to the treated skin area. A presently preferred backing sheet is an ethyl vinyl acetate film having a thickness of about 20 to about 100 μm, for example Mediflex® 1200 of Mylan Technologies, Inc.

Transdermal delivery can be achieved by several means. Medicinal bandage type delivery devices use a membrane-controlled system or a matrix system in which the active ingredient reservoir is in the adhesive layer (S. Venkatraman, N. Davar, A. Chester, and L. Kleiner in “An Overview of Controlled Release Systems” pp. 445-452 in Handbook of Pharmaceutical Controlled Release Technology ed Donald L. Wise, Marcel Dekker, Inc., NY 2000.) Alternate means include spraying on the active ingredient from a liquid such that due to evaporation of the liquid the active ingredient is in a more concentrated form on the skin. This type of embodiment is exemplified by U.S. Pat. No. 5,958,379 and is included herein. The active ingredient could also be applied to the skin in the form of an ointment, cream, lotion, solution or plaster or by other means known by those experienced in the art.

In a specific embodiment for transdermal administration, the active ingredient can be contained in a matrix from which it is released in the desired gradual, constant and controlled manner. The permeability of the matrix during the release of the active ingredient is based on diffusion. A system of this type is described in the U.S. Pat. No. 4,769,028 and is included herein by reference. It consists of an impermeable backing layer, an active compound reservoir associated with and made of a polymer compound, a contact adhesive layer that is permeable to the active ingredient, associated with the reservoir and a protective layer covering the contact-adhesive layer, which can be removed for use. Other embodiments include reservoir layers that have a high intrinsic tackiness that can be used simultaneously as the contact-adhesive layer.

One useful embodiment is a method of making a pressure sensitive adhesive matrix patch for transdermal delivery using hydrophilic active ingredients and their salts as described in U.S. Pat. No. 6,365,178 B1, which is hereby incorporated by reference. The steps of making one embodiment of the matrix transdermal device are:

a. dissolving an effective amount of hydrophilic active ingredient with an aqueous dispersion, comprising a water phase, of a hydrophobic pressure sensitive adhesive and optionally a permeation enhancer, to form a mixture;
b. film casting the mixture and evaporating the water phase to obtain a hydrophobic pressure sensitive adhesive matrix film having the active ingredient fully dissolved therein and having first and second surfaces, thereof; and
c. laminating a release liner to the first surface of the matrix film and a substantially drug-impermeable backing layer to the second surface.
Preferably the water-based adhesive is an acrylic copolymer, ethylene-vinyl acetate copolymer, or polyisobutylene polymer or copolymer that is a two-phase dispersion of water-insoluble polymer particles in water. The pressure sensitive adhesives must also be pharmaceutically acceptable.
The backing layer, which adheres to the active ingredient containing adhesive layer serves as the upper layer of the patch during use and functions as the primary structural element of the transdermal device. The backing layer is made of a sheet of film of a preferably flexible elastomeric material that is substantially impermeable to the active ingredient and any permeation enhancer that may be present. The backing layer is typically about 0.001-0.004 inch in thickness and is preferably of a material that permits the transdermal device to be worn comfortably on any skin area. Examples of polymers useful for the backing layer may include, but are not limited to, polyethylene, polypropylene, polyesters, polyurethanes, polyethylene vinyl acetate, polyvinylidene chloride, block copolymers such as PEBAX, and the like. The backing layer can also comprise laminates of one or more of the foregoing polymers.

The release liner is a disposable element that serves only to protect the transdermal device prior to application to the skin. Typically the release liner is formed from a material impermeable to the active ingredient, permeation enhancer and any other components of the transdermal device, and is easily strippable form the pressure sensitive adhesive. Release liners can generally be made of the same materials as the backing layer.

The active ingredient adhesive matrix layer can, in addition to the adhesive, active ingredient and optional permeation enhancer, also contain other optional ingredients, such as thickeners, excipients, emulsifiers, tackifiers, preservatives, defoamers, antioxidants, surfactants and the like, which are nontoxic and do not interfere with delivery of the active ingredient.

Other embodiments of the medicinal bandage include a rate controlling membrane and/or the active ingredient reservoir dispersed in a solid carrier, semi-solid carrier or ointment and are exemplified by U.S. Pat. No. 4,752,478 and is hereby incorporated by reference. The rate-controlling membrane can be a microporous membrane and can be made of any porous material allowing the passage of the active ingredient such as but not limited to micorporous polypropylene, microporous nylon, microporous polycarbonate. Other materials that can be used as rate-controlling membrane include ethylene vinyl acetate copolymers and polyethylene as disclosed in U.S. Pat. No. 4,588,580 and other materials as disclosed in U.S. Pat. No. 3,797,494 which are both hereby incorporated by reference.

Another embodiment of the rate-controlling membrane is exemplified by U.S. Pat. No. 6,375,978 B1 and is hereby incorporated by reference. In this embodiment the rate-controlling membrane may be pretreated by an annealing process to provide consistent membrane functionality over time and elevated storage conditions.

For a particular membrane material, the rate control of the transdermal drug delivery device is dependent on but not limited to the composition, pore size, and thickness of the rate-controlling membrane, the viscosity of the active ingredient formulation and any diffusive medium that can impregnate the pores.

The transdermal device may be comprised of an active ingredient reservoir layer between a backing layer and a contact adhesive layer, wherein the rate controlling membrane is on the skin-proximal side of the active ingredient reservoir layer. The drug reservoir may optionally include permeation enhancers and/or other excipients. An alternate embodiment of this device has a separate permeation enhancer reservoir which is located on the skin proximal side of the backing layer and separated from the active ingredient reservoir which is proximal to the skin by the rate-controlling membrane. The membrane material may be selected from but not limited to the following materials: ethylene vinyl acetate copolymers, polyethylene, copolymers of ethylene, polyolefins including ethylene oxide copolymers such as Engage® (DuPont Dow Elastomers), polyamides, cellulosic materials, polyurethanes, polyether blocked amides copolymers such as PEBAX® (Elf Atochem North America, Inc.) and polyvinyl acetate.

The active ingredient and/or permeation enhancer reservoir(s) can be a gel or a polymer and may comprise an aqueous or non-aqueous composition. Suitable materials used in the reservoir include, but are not limited to gelled mineral oil, polyisobutylene, aluminum stearate, propylene glycol, fatty acid esters, natural and synthetic rubbers or other polymeric material, silicone fluids, polysiloxanes, polyacrylates, ethylene vinyl acetate copolymers, hydroxyethylene cellulose, hydroxypropyl cellulose, hydroxypropylmethylcellulose and petroleum jelly. Permeation enhancers include, but are not limited to ethanol and other higher alcohols, N-decylmethylsulfoxide (nDMS), polyethylene glycol monolaurate, dilaurate and related esters, glycerol mono-oleate and related mono, di and trifunctional glycerides, diethyl toluamide, Azone® (a product of Nelson Research Corp.,) N,N-dimethyl lauramide, N,N-dimethyl lauramine oxide.

In addition to the active ingredient(s) and permeation enhancer(s) the transdermal device may include stabilizers, dyes, pigments, inert fillers, tackifiers, excipients and other components of transdermal delivery devices as are known in the art.

In one particular embodiment the rate controlling membrane is subjected to an annealing process to maintain a permeability within a preferred range even after the transdermal device is exposed to elevated temperatures. A preferred embodiment uses a rate-controlling membrane comprised of ethylene vinyl acetate (EVA) copolymer. The desired membrane permeability is achieved by proper selection of the vinyl acetate (VA) content of the copolymer in addition to the proper annealing conditions. Preferred annealing conditions include for example an annealing temperature of about 45-75° C., most preferred about 52-72° C., for a period of about 1-72 hours, most preferred about 2-36 hours, and a VA content of 4-18%, most preferred about 5-12%.

In order to facilitate transport of hydrophilic active ingredients through the skin other transdermal embodiments may be applied. Some such methods include iontophoresis, electro-osmosis and electroporation as described by A. K. Banga in the chapter “Electrically Assisted Transdermal Delivery of Drugs” pp. 567-581 and by T. Chen, R. Langer and J. C. Weaver in the chapter “Transdermal Drug Delivery by Skin Electroporation” pp. 597-605 and S. Mitragotri, R. Langer and J. Kost in the chapter “Enhancement of Transdermal Transport Using Ultrasound in Combination with Other Enhancers” pp. 607-616 and E. R. Scott, J. B. Phipps, J. R. Gyory and R. V. Padmanabhan in the chapter “Electrotransport Systems for Transdermal Delivery: A Practical Implementation of Iontophoresis” pp. 617-659 in the Handbook of Pharmaceutical Controlled Release Technology, Donald L. Wise, ed., Marcel Dekker, Inc., NY 2000 and is hereby incorporated by reference. One such embodiment is a transdermal delivery system by Altea Therapeutics which forms micropores in the skin as exemplified by U.S. Pat. No. 6,183,434 B1 and is hereby incorporated by reference. An electrotransport and an iontophoretic system are exemplified by U.S. Pat. No. 6,725,090 B1 and U.S. Pat. No. 6,317,629 B1, respectively and are both hereby incorporated by reference.

Another embodiment of a transdermal dosage form of the active ingredient is a liquid formulation that is sprayable in droplets as described in U.S. Pat. No. 5,958,379 which is hereby incorporated by reference. After being sprayed the composition forms a preparation within a short time, preferably within a time of less than 4 seconds, on the sprayed body surface, particularly on the sprayed skin or mucous membrane, whereby, compared to the original liquid composition, the preparation is formed to have a higher concentration of the active ingredient and which contains the active ingredient in a finely divided way. The composition can be applied by spraying a specific volume to achieve the desired dose.

In order to achieve fast evaporation, such that the liquid composition forms the preparation on the body surface within 4 second, more preferably with a time of less than 2 seconds and most preferable within a time of between 1 second and 0.01 seconds, the composition preferably comprises in addition to the active ingredient such liquids that allow the spraying of droplets with a diameter between 1 micron and 1,000 microns, preferably in droplets having diameters between 10 microns and 100 microns. These additives can be used to affect the surface tension, the zeta potential and viscosity. In one embodiment the liquid composition comprises a viscosity of between 1 mPas and 100 mPas, preferably between 5 mPas and 25 mPas before spraying. The liquid composition is formed such that the active ingredient of the preparation formed on the sprayed body surface is between 25% and 500%, preferably between 50% and 150%, higher that the concentration of the active ingredient of the composition before spraying.

Possible additional ingredients of the liquid formulation include a gel-forming agent. Gel-forming agents may include, but are not limited to, synthetic gel-forming agents such as polymers, particularly polyacrylates, cellulose derivatives, polyvinylalcohols and/or polyvinylacetate. The gel-forming agents can be a natural gel-forming agent, preferably phospholipids or a phospholipids mixture and particularly phospholipids and/or mixture isolated from soybeans, sunflowers and eggs. A particular phospholipid is phosphatidylcholine.

The liquid composition is in the form of a dispersion, a solution and/or a suspension. The composition contains a liquid as a liquid mixture, a pharmaceutically acceptable organic solvent or solvent mixture and/or water. Preferably the liquid composition contains an organic solvent, or as an organic solvent mixture, one or several alcohols which can easily be evaporated. The low molecular weight and easily evaporatable alcohols may include, but are not limited to, ethanol, propanol-1, propanol-2 and/or butanol. The liquid composition may also include propylene glycol alone or in a mixture with the afore mentioned alcohols as organic solvents. A particular embodiment includes a dispersion containing liposomes.

The liquid composition may include other ingredients such as but not limited to preservatives or buffers.

A spraying device is used which preferably operates without a blowing agent and which comprises a pump sprayer, whereby 20 to 300 microliters of the liquid composition per spraying, particularly with a droplet size of between 1 micron and 1,000 microns, preferably with a droplet size of between 10 microns and 100 microns are emitted by means of a suitable spraying nozzle.

Other features and embodiments of the invention will become apparent from the following examples, which are given for illustration of the invention rather than for limiting its intended scope.

### Example 1

As a way of measuring the skin permeation properties of active ingredient, a Franz diffusion cell was provided utilizing a human cadaver skin membrane and a receptor fluid such as 1.0M phosphate buffered saline. The receptor compartment of the Franz diffusion cell was filled with the receptor fluid and the diffusion cell was maintained at 34.5° C. Human cadaver skin was cut out to provide a membrane of 1.767 cm² surface area. The amount of active ingredient that had permeated through the membrane by various times in a 6 to 48 hour period was determined by HPLC analysis of the receptor fluid. Each test was conducted in several replicates. The average calculated skin flux and average amount penetrating per square centimeter after 24 and 48 hours for Formulation I and II are shown in Table 1. These experimental results using the Franz diffusion cell methodology indicate that transdermal delivery of this compound is feasible based on theoretical skin fluxes for the active ingredient of 0.3 to 5 μg/cm²/hr.

### Formulation Preparation

Formulation I: One gram of varenicline tartrate was dissolved into 5 mL of phosphate buffer (pH 7.0). This formulation was mixed using a vortex mixer and sonicated for 5 minutes. The solution was placed in an amber bottle to limit its exposure to light. The varenicline tartrate did not completely dissolve and the pH of the final solution was 3.41. The resulting saturated solution was used in the in-vitro Franz diffusion cell skin permeation study without filtering to determine the ability of the compound itself to penetrate the skin.

Formulation II: One hundred milligrams of varenicline tartrate were placed in 10 mL of a 50/50 v/v combination of propylene glycol and phosphate buffer solution (pH=7.0). This formulation was mixed using the vortex mixer and sonicated for 5 minutes. The resulting formulation was a 10 mg/mL non-saturated solution of varenicline tartrate. The actual parent varenicline concentration in this formulation was 5.85 mg/mL. The pH of the final solution was 4.94. This formulation was tested to determine the flux when organic components are also present, in this case propylene glycol was used since it could potentially be used as a solvent and/or a permeation enhancer in either a reservoir style or matrix type device.

### Franz Diffusion Cell Experimental Parameters

Apparatus: Hansen Automated Franz diffusion cell sampling system

### Cell Volume: 7.0 mL

Receptor Solution Phosphate buffered saline (1.0 M))
Membrane Samples Human Cadaver skin, abdomen, male
Test Formulations: Formulation I: Saturated phosphate buffer solution
Formulation II: 1% varenicline tartrate in 50/50 propylene glycol/phosphate buffer solution
Dose: 500 μL over a 1.767 cm² area; occluded
Duration: 48 hours

### Temperature: 34.5° C.

Sample volume: 1.0 mL
Rinse volume: 1.5 mL

### Sample Analysis: LC/MS/MS

TABLE 1
Calculated Skin Flux and Amount Penetrating for Formulations I and II

| | Ave | Ave Amt | Ave Amt |
|---|---|---|---|
| | Skin Flux | Penetrating @ | Penetrating @ |
| | (μg/cm²/hr) | 24 hrs (μg/cm²) | 48 hrs (μg/cm²) |
| Formulation I | 12.56 ± 12.32 | 267 ± 259 | 581 ± 543 |
| Formulation II | 2.41 ± 0.26 | 37.9 ± 16.0 | 49.3 ± 20.0 |

### Example 2

### Matrix Type Transdermal

The active ingredient is mixed with the aqueous dispersion of NACOR 72-9965 (hydrophobic acrylic copolymer from National Starch) to achieve a 2% (w/w) concentration of active ingredient in the dried film after film casting. The adhesive mixture is cast on a release coated polymer film (Rexam Release Technologies; W. Chicago, Ill.) and is dried at 60° C. in a convective oven and cut to achieve a 2 mgA dose of the active ingredient. The dried film is laminated to a polyester film laminate (SCOTCHPACK #1012, 3M Pharmaceuticals; St. Paul, Minn.).

### Example 3

### Matrix Type Transdermal Systems

(1) The base form or salt forms of the active ingredient is dissolved or dispersed in a polyacrylate solution, such as Duro-Tak® 387-2052 adhesive. Appropriate solvent, enhancer and/or filler is added in the adhesive dispersion, and mixed well. Air is removed from the resulting mixture and laminated on a release liner, such as Medirelease® 2228, to form a coating thickness of 0.5-2 mm. The adhesive layer is dried at room temperature for 5-10 min and then at 40-80° C. for 15-30 min to remove all volatile solvents. A backing sheet, such as Mediflex® 1200, is coated on the adhesive side. The resulting patches of a desired size are stored in sealed packages.

(2) The base form or salt forms of the active ingredient is dissolved or dispersed in a polyisobutylene (PIB) based adhesive, such as Duro-Tak® 87-6173. The following procedures are similar to those described in the previous section.

(3) The base form or salt forms of the active ingredient is dissolved or dispersed in a silicone-based adhesive, such as Bio-PSA® 7-4302. The following procedures are similar to those described in the previous section.

### Example 4

### Rate-Controlling Membrane Annealed for Better Stability

The active ingredient is added to a mixture of 95% ethanol and purified water. 2% of hydroxyethyl cellulose gelling agent is added slowly to the active solution with stirring and is mixed until a smooth gel is obtained. A 0.05 mm thick contact adhesive layer is formed on a release liner for the system by solution casting an amine resistant silicone medical adhesive (XCF 2992, Dow Corning, Midland, Mich.) onto the polyester film from a solution in heptane.

An annealed or non-annealed rate-controlling membrane comprised of EVA (9% VA) is pressure laminated to the exposed adhesive to obtain a thickness on the order of 2-3 mils. (If annealed, the rate-controlling membrane is kept at 60° C. for a period of about 24 hours and then cooled to ambient conditions for about 48 hours before being pressure laminated to the adhesive layer.)

A backing layer comprised of a multilaminate of polyester, polyethylene, aluminum, polyester and EVA (Scotchpak 1220, 3M Co., St. Paul, Minn.) is used to pouch the active aqueous gel between the backing layer and the release liner/adhesive/rate-controlling membrane using a rotary heat-seal machine. The sealed pouch is die cut in a size to provide the desired amount of active ingredient on the order of 0.5-6.0 mg.

### Example 5

### Sprayable Liquid

The pharmaceutical composition is manufactured with the following ingredients:

10 g phospholipidic gel-forming agent

16 g ethanol
1 g active ingredient

0.5 g phosphate buffer

distilled water to obtain 100 g of the liquid composition

The 10 g of phospholipidic gel-forming agent is hydrated in about 30% by volume of the total amount of water. The active ingredient and ethanol is added to the hydrated gel-forming agent and mixed in a high pressure homogenizer. The buffer and remaining water is added. Through homogenization an average vesicle diameter of about 100 nm is obtained.

What is claimed is:

1. A transdermal composition comprising varenicline or its pharmaceutically acceptable salt or prodrug form.

2. The transdermal composition of claim 1, further comprising a backing sheet having opposing surfaces that are respectively distal and proximal to the skin when applied and a coating on the proximal surface of the backing sheet.

3. The transdermal composition of claim 2, wherein the coating comprises (a) an adhesive and (b) varenicline or its pharmaceutically acceptable salt or prodrug form in a water-soluble form in a therapeutically effective total amount and dispersed in a matrix.

4. The transdermal composition of claim 3, wherein the matrix comprises an adhesive.

5. The transdermal composition of claim 3, wherein the coating comprises a first reservoir layer which contains the varenicline adjacent to the backing sheet, and a second adhesive layer that is proximal to the skin when applied, wherein, optionally, a membrane that permits passage of the varenicline is present between the reservoir layer and the adhesive layer.

6. The transdermal composition of claim 5, wherein the coating further comprises one or more skin permeation enhancers, stabilizers, dyes, pigments, inert fillers, tackifiers, gel forming agents and optional antioxidants.

7. The transdermal composition of claim 6, wherein the permeation enhancers are chosen from ethanol and other higher alcohols, N-decylmethylsulfoxide (nDMS), polyethylene glycol monolaurate, dilaurate and related esters, glycerol mono-oleate and related mono-, di- and trifunctional glycerides, diethyl toluamide, 1-Dodecylazacycloheptan-2-one, N,N-dimethyl lauramide, N,N-dimethyl lauramine oxide and mixtures thereof.

8. The transdermal composition of claim 3, wherein the adhesive is selected from an acrylic copolymer, ethylene-vinyl acetate copolymer, and polyisobutylene polymer or copolymer that is a two-phase dispersion of water-insoluble polymer particles in water.

9. The transdermal composition of claim 1, wherein varenicline is present in an amount which releases from about 0.1 mgA to about 6 mgA per day.

10. The transdermal composition of claim 9, wherein varenicline is present in an amount which releases from about 0.5 mgA to 4 mgA per day.

11. A method for reducing nicotine addiction or aiding in the cessation or lessening of tobacco use in a subject, comprising transdermal administration to a subject an amount of varenicline that is effective in reducing nicotine addiction or aiding in the cessation or lessening of tobacco use.

12. The method of claim 11, wherein the varenicline is present as varenicline tartrate.

13. A method of making a pressure sensitive adhesive matrix patch for transdermal delivery of varenicline by the steps consisting of:
(a) dissolving an effective amount of varenicline in an aqueous dispersion, comprising a water phase, a hydrophobic pressure sensitive adhesive and optionally a permeation enhancer, to form a mixture;
(b) film casting the mixture and evaporating the water phase to obtain a hydrophobic pressure sensitive adhesive matrix film having first and second surfaces, the varenicline being fully dissolved therein; and,
(c) laminating a release liner to the first surface of the matrix film and a substantially drug-impermeable backing layer to the second surface, thereby providing a pressure-sensitive adhesive matrix patch for transdermal delivery of varenicline.

14. A sprayable liquid for preparation of a transdermal composition of varenicline, or a pharmaceutically acceptable salt thereof. comprising phospholipidic gel-forming agent, ethanol, varenicline, phosphate buffer, and water.

15. The sprayable liquid of claim 12, further comprising a phospholipidic gel-forming agent, ethanol, phosphate buffer, and water.

16. The transdermal composition of claim 6, wherein the varenicline is varenicline free base and an antioxidant is present.
